(19) United States
(12) Patent Application Publication
(10) Pub. No.: US 2010/0115651 A1
(43) Pub. Date: May 6, 2010

(54) Mutant Allele of Watercress

(75) Inventor: William L. Summers, Vero Beach, FL (US)

Correspondence Address: JONDLE & ASSOCIATES P.C., 858 HAPPY CANYON ROAD SUITE 230, CASTLE ROCK, CO, 80108, US

(73) Assignee: B&W QUALITY GROWERS, INC., Fellsmere, FL (US)

(21) Appl. No.: 12/611,276

(22) Filed: Nov. 3, 2009

Related U.S. Application Data
(60) Provisional application No. 61/111,247, filed on Nov. 4, 2008.

(51) Int. Cl. A01H 5/00; A01H 1/02; C12N 15/82; C12N 15/31

(52) U.S. Cl. 800/260; 800/298; 800/278; 800/300; 800/279; 800/302; 800/301; 800/281; 800/284

(57) ABSTRACT

The present invention relates to a watercress plant, seed, variety and hybrid. More specifically, the invention relates to a watercress plant having a mutant allele designated BWRW which results in a watercress plant having red-pigmented leaves and/or apical stems. The invention also relates to crossing inbreds, varieties and hybrids containing the BWRW allele to produce watercress plants having red-pigmented leaves and/or apical stems.

### CROSS REFERENCE

This application claims the benefit of U.S. Provisional Application No. 61/111,247 filed on Nov. 4, 2008, the specification of which is hereby incorporated in its entirety.

### BACKGROUND OF THE INVENTION

The present invention relates to an allele of watercress designated “BWRW”, which results in watercress plants with red-pigmented leaves. The present invention also relates to a watercress seed, a watercress plant and parts of a watercress plant, a watercress variety and a watercress hybrid which comprise the mutant allele. In addition, the present invention is directed to transferring the BWRW allele in the watercress plant to other watercress varieties and species and is useful for producing new types and varieties of red-pigmented watercress plants. All publications cited in this application are herein incorporated by reference.

The present invention relates to a new and distinctive watercress mutant allele, designated “BWRW”. There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety or hybrid an improved combination of desirable traits from the parental germplasm. These important traits may include higher yield, better field performance, resistance to diseases and insects, better stems, leaves and roots, tolerance to drought, cold and/or heat, and better culinary quality.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection.

These processes, which lead to the final step of marketing and distribution, usually take from eight to 12 years from the time the first cross is made. Therefore, development of new cultivars is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of watercress plant breeding is to develop new, unique and superior watercress inbreds and hybrids. The breeder initially selects and crosses two or more parental lines, followed by repeated selfing and selection, producing many new genetic combinations. The breeder can theoretically generate billions of different genetic combinations via crossing, selfing and mutations. The breeder has no direct control at the cellular level. Therefore, two breeders will never develop the same line, or even very similar lines, having the same watercress traits.

Each year, the plant breeder selects the germplasm to advance to the next generation. This germplasm is grown under unique and different geographical, climatic and soil conditions, and further selections are then made, during and at the end of the growing season. The varieties which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce the same line twice by using the exact same original parents and the same selection techniques. This unpredictability results in the expenditure of large research monies to develop superior new watercress varieties.

The development of commercial watercress hybrids requires the development of homozygous inbred lines, the crossing of these lines, and the evaluation of the crosses. Pedigree, backcross or recurrent selection breeding methods are used to develop lines from breeding populations. Breeding programs combine desirable traits from two or more lines or various broad-based sources into breeding pools from which mutant alleles are developed by selfing and selection of desired phenotypes. The new inbreds are crossed with other inbred lines and the hybrids from these crosses are evaluated to determine which have commercial potential.

Pedigree breeding is used commonly for the improvement of self-pollinating crops or inbred parents of cross-pollinating crops. Two parents which possess favorable, complementary traits are crossed to produce an F₁. An F₂ population is produced by selfing one or several F₁'s or by intercrossing two F₁'s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars or new parents for hybrids.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or inbred which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In a multiple-seed procedure, breeders commonly harvest two or more seeds from each plant in a population and bulk them to form a bulk sample. Part of the bulk is used to plant the next generation and part is put in reserve. The procedure has been referred to as modified single-seed descent or the “bulk” technique.

The multiple-seed procedure has been used to save labor at harvest. It is considerably faster to extract seeds with a machine than to remove one seed from each by hand for the single-seed procedure. The multiple-seed procedure also makes it possible to plant the same number of seeds of a population each generation of inbreeding. Enough seeds are harvested to make up for those plants that did not germinate or produce seed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Isozyme Electrophoresis and RFLPs have been widely used to determine genetic composition. Shoemaker and Olsen, (Molecular Linkage Map of Soybean (Glycine max L. Men.) p 6.131-6.138 in S. J. O'Brien (ed) Genetic Maps: Locus Maps of Complex Genomes, Cold Spring Harbor Laboratory Press, Cold Spring Harbor, N.Y., (1993)) developed a molecular genetic linkage map that consisted of 25 linkage groups with about 365 RFLP, 11 RAPD, three classical markers and four isozyme loci. See also, Shoemaker, R. C., RFLP Map of Soybean, p 299-309, in Phillips, R. L. and Vasil, I. K., eds. DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225, 1997.) SNPs may also be used to identify the unique genetic composition of the invention and progeny varieties retaining that unique genetic composition. Various molecular marker techniques may be used in combination to enhance overall resolution.

Molecular markers, which includes markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding is another method of introducing new traits into pumpkin and squash varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogues like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

Descriptions of other breeding methods that are commonly used for different traits and crops can be found in one of several reference books (e.g., Allard, 1960; Simmonds, 1979; Sneep et al., 1979; Fehr, 1987).

Proper testing should detect any major faults and establish the level of superiority or improvement over current cultivars. In addition to showing superior performance, there must be a demand for a new cultivar that is compatible with industry standards or which creates a new market. The introduction of a new cultivar will incur additional costs to the seed producer, the grower, processor and consumer for special advertising and marketing, altered seed and commercial production practices, and new product utilization. The testing preceding release of a new cultivar should take into consideration research and development costs as well as technical superiority of the final cultivar.

Watercress is also known as Nasturtium officinale and it belongs to the family Cruciferae. Watercress is native to Europe and Asia, common in Great Britain and widely naturalized in the United States and Canada. It has also been introduced into the West Indies and South America. It is reported that Nicholas Messier first grew watercress in Erfurt, Germany in the middle of the 16th century. English cultivation started in early 1800, when a farmer near London began to grow watercress for use in salads. It was not long before its popularity spread. Today the crisp green sprigs of watercress are commonly eaten out of hand, combined with other tender greens in salads and used as a garnish on hot and cold dishes.

The foregoing examples of the related art and limitations related therewith are intended to be illustrative and not exclusive. Other limitations of the related art will become apparent to those of skill in the art upon a reading of the specification.

### SUMMARY OF THE INVENTION

The following embodiments and aspects thereof are described in conjunction with systems, tools and methods which are meant to be exemplary, not limiting in scope. In various embodiments, one or more of the above-described problems have been reduced or eliminated, while other embodiments are directed to other improvements.

According to the invention, there is provided a new mutant allele designated “BWRW”. This invention thus relates to a watercress seed, a watercress plant, a part of a watercress plant, a watercress variety, a watercress hybrid, and to a method for producing a watercress plant. More specifically, the invention relates to a mutant allele designated BWRW which produces a watercress plant with red-pigmented leaves and/or apical stems.

Another aspect of the invention relates to any watercress seed, plant, or part thereof, having the mutant allele BWRW.

In another aspect, the present invention provides regenerable cells for use in tissue culture. The tissue culture will preferably be capable of regenerating plants having the physiological and morphological characteristics of the foregoing watercress plant, and of regenerating plants having substantially the same genotype as the foregoing watercress plant. Preferably, the regenerable cells in such tissue cultures will be embryos, protoplasts, meristematic cells, callus, pollen, leaves, anthers, pistils, stems, petioles, roots, root tips, fruits, seeds, flowers, cotyledons, hypocotyls or the like. Still further, the present invention provides watercress plants regenerated from the tissue cultures of the invention.

Another aspect of the invention is to provide methods for producing other watercress plants derived from a watercress plant having the BWRW allele. Watercress lines derived by the use of those methods are also part of the invention.

The invention also relates to methods for producing a watercress plant containing in its genetic material one or more transgenes and to the transgenic watercress plant produced by that method.

The invention further provides methods for developing watercress plants in a watercress plant breeding program using plant breeding techniques including recurrent selection, backcrossing, pedigree breeding, restriction fragment length polymorphism enhanced selection, genetic marker enhanced selection and transformation. Watercress seeds, plants and parts thereof produced by such breeding methods are also part of the invention.

In addition to the exemplary aspects and embodiments described above, further aspects and embodiments will become apparent by study of the following descriptions.

### DEFINITIONS

In the description and tables that follow, a number of terms are used. In order to provide a clear and consistent understanding of the specification and claims, including the scope to be given such terms, the following definitions are provided:

Allele. An “allele” is any of one or more alternative form of a gene, all of which alleles relates to one trait or characteristic. In a diploid cell or organism, the two alleles of a given gene occupy corresponding loci on a pair of homologous chromosomes. The BWRW allele of the present invention may refer to one or more alleles.

Backcross. “Backcross” is a breeding method in which a breeder repeatedly crosses hybrid progeny back to one of the parents, for example, a first generation hybrid F₁ with one of the parents of the F₁ hybrid.

Bunch. A “bunch” of watercress 17.15 cm (6.75 in) long weighs about 0.25 lb. A commercial bunch of watercress may range from 17.15 cm (6.75 in) to 60.96 cm (24.0 in) in length and may increase in weight as the stems get longer.

BWRW. “BWRW” refers to the mutant allele or alleles of the present invention that results in red-pigmented leaves in watercress plants.

Chromosome doubling agent. A “chromosome doubling agent” is any one of a number of mitotic inhibitors, including colchicine, oryzalin, trifluralin, amiprophos-methyl, and N₂O gas.

Colchicine. Colchicine is an alkaloid prepared from the corms and seeds of Colchicum autumnale, the autumn crocus. Normally after a cell has copied its chromosomes in preparation for cell division, the spindle forms, attaches to the chromosomes and moves the chromosomes to opposite sides of the cell. The cell then divides with a set of chromosomes in each of the daughter cells. Colchicine suppresses cell division by inhibiting formation of the spindle microtubules which prevents the cell from distributing the two copies of the chromosomes to opposite sides of the cell. The cell then fails to divide.

Diploid. “Diploid” means having two sets or a pair of chromosomes.

Diploid plants. “Diploid plants” means plants or transplants derived from planting diploid seeds or from micro propagation.

Essentially all the physiological and morphological characteristics. A plant having “essentially all the physiological and morphological characteristics” means a plant having the physiological and morphological characteristics, except for the characteristics derived from the converted gene or genes.

Gene converted (conversion). “Gene converted” (or conversion) plant refers to plants which are developed by backcrossing wherein essentially all of the desired morphological and physiological characteristics of an inbred are recovered in addition to the one or more genes transferred into the plant via the backcrossing technique or wherein plants are developed with one or more genes or loci via genetic engineering or mutation.

Gene silencing. “Gene silencing” means the interruption or suppression of the expression of a gene at the level of transcription or translation.

Genotype. “Genotype” refers to the genetic constitution of a cell or organism.

Haploid. “Haploid” means having the same number of sets of chromosomes as a germ cell or half as many as a somatic cell.

Hexaploid. “Hexaploid” means having six times the haploid number of chromosomes in the cell nucleus.

Hexploid plant. “Hexaploid plant” means plants or transplants derived from hexaploid seeds, cuttings, embryos or from micro propagation.

Linkage. “Linkage” refers to a phenomenon wherein alleles on the same chromosome tend to segregate together more often than expected by chance if their transmission was independent.

Linkage disequilibrium. “Linkage disequilibrium” refers to a phenomenon wherein alleles tend to remain together in linkage groups when segregating from parents to offspring, with a greater frequency than expected from their individual frequencies.

Locus. A “locus” confers one or more traits such as, for example, male sterility, herbicide tolerance, insect resistance, disease resistance, modified fatty acid metabolism, modified carbohydrate metabolism and modified protein metabolism. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

Plant. “Plant” includes plant cells, plant protoplasts, plant cells of tissue culture from which watercress plants can be regenerated, plant calli, plant clumps and plant cells that are intact in plants or parts of plants such as pollen, flowers, seed, leaves, stems, roots and the like.

Ploidy. “Ploidy” refers to the number of single sets of chromosomes in a cell or organism.

Quantitative trait loci (QTL). “Quantitative trait loci (QTL)” refer to genetic loci that control to some degree numerically representable traits that are usually continuously distributed.

Regeneration. “Regeneration” refers to the development of a plant from tissue culture.

Red-pigmented. “Red-pigmented” as used herein means any part of an immature or mature leaf or apical stem having colors that range from RHS N34A to RHS 187D. More particularly, the red-pigmented colors include RHS N34A, RHS 45A, RHS 45B, RHS 45C, RHS 45D, RHS 46A, RHS 46B, RHS 46C, RHS 46D, RHS 47A, RHS 47B, RHS 47C, RHS 47D, RHS 53A, RHS 53B, RHS 53C, RHS 53D, RHS 58A, RHS 59A, RHS 59B, RHS 59C, RHS 60A, RHS 60B, RHS 60C, RHS 61A, RHS 63A, RHS 64A, RHS 64B, RHS 77A, RHS N77A, RHS N77B, RHS 79A, RHS 79B, RHS 79C, RHS 79D, RHS N79A, RHS N79B, RHS N79C, RHS N79D, RHS 83A, RHS N92A, RHS 181A, RHS 181B, RHS 181C, RHS 181D, RHS 182A, RHS 182B, RHS 182C, RHS 182D, RHS 183A, RHS 183B, RHS 183C, RHS 183D, RHS 184A, RHS 184B, RHS 184C, RHS 184D, RHS 185A, RHS 185B, RHS 185C, RHS 185D, RHS 186A, RHS 186B, RHS 186C, RHS 186D, RHS N186A, RHS N186B, RHS N186C, RHS N186D, RHS 187A, RHS 187B, RHS 187C, and RHS 187D.

Triploid. “Triploid” means having three times the haploid number of chromosomes in the cell nucleus.

Triploid plants. “Triploid plants” means plants or transplants derived from triploid seeds, cuttings, embryos or from micro propagation.

Yield. The term “Yield” is typically defined when used. Yield can mean the number of bunches of watercress per acre where, on average, 4 bunches equal approximately a pound. Alternatively, if watercress is harvested loose, yield may be measured in pounds (lbs) or kilograms (Kg) per acre.

### DETAILED DESCRIPTION OF THE INVENTION

The present invention relates to a new allele designated “BWRW” in the genus Nasturtium that is phenotypically expressed in a watercress plant having red-pigmented leaves and/or apical stems. The present invention also relates to a watercress seed, a watercress plant and plant parts, a watercress variety and a watercress hybrid which comprise the new BWRW allele. The present invention also relates to a method of producing the disclosed watercress plants and seeds.

The allele of the present invention is readily transferred between the deposited cultivar and its related cultivars.

A plant of the present invention can be obtained by crossing a plant containing the claimed mutant allele with any watercress cultivar lacking the allele. The plant containing the allele can be any watercress variety including a cultivar in which the factor has been previously genetically fixed.

Other breeding schemes can be used to introduce the BWRW allele into the desired cultivar. The particular scheme used is not critical to the invention, so long as the allele is stably incorporated into the genome of the cultivar. For example, a marker gene can be used. A nucleic acid probe which hybridizes to the marker gene can be used to identify the desired plants in the F₁ generation.

The BWRW allele will advantageously be introduced into varieties that contain other desirable genetic traits such as resistance to disease, drought tolerance, heat and/or cold tolerance, and the like.

The invention also relates to methods for producing a watercress plant containing in its genetic material one or more transgenes and to the transgenic watercress plant produced by that method.

In another aspect, the present invention provides for single gene converted plants of BWRW. The single transferred gene may preferably be a dominant or recessive allele. Preferably, the single transferred gene will confer a trait such as male sterility, herbicide resistance, insect resistance, resistance for bacterial, fungal, or viral disease, male fertility, early maturity, enhanced nutritional quality, and enhanced flavor. The single gene may be a naturally occurring watercress gene or a transgene introduced through genetic engineering techniques.

The invention further provides methods for developing watercress plants in a watercress plant breeding program using plant breeding techniques including recurrent selection, backcrossing, pedigree breeding, restriction fragment length polymorphism enhanced selection, genetic marker enhanced selection, chromosome doubling and transformation. Seeds, watercress plants, and parts thereof produced by such breeding methods are also part of the invention.

Watercress is a perennial plant and is generally produced for human consumption. Watercress is typically grown in rectilinear ponds with flowing water whose source is tested frequently for the presence of harmful pathogens. The flowing water may flow through the bed and exit or it may be re-circulated to flow through multiple times. Companies may also add a proprietary blend of fertilizer elements to the irrigation water in the flow-through system. Each pond will have a base to support watercress axil root growth. The base composition may be natural soil, which is commonly used in the United States, soil overlaid with pebbles, which is commonly used in England, or various plastic compounds overlaid with a more porous material that roots can hook on to.

Watercress may be propagated via vegetative cuttings strewn randomly over the pond's surface or the crop can be seeded onto a growing medium overlaid onto plastic. When the seedlings are of appropriate size they are scooped up and flung randomly onto the pond's surface. Additionally, the crop can be direct seeded by a precision seeder or drill. Typically the first crop reaches a harvestable stage in about 7 weeks with subsequent harvests occurring at 4-week intervals. Harvest can occur by hand making a bunch in the field and placing a rubber band around the bunch and trimming the end to the desired length. The desired length varies in the United States from 17.15 cm (6.75 in) to about 60.96 cm (24 in). Bunches are transported to the packing house for washing, packing, and refrigeration. Watercress may also be harvested by machine to yield a loose product that is packaged in different sized packages.

Watercress is often used to make tea or to make a stock base for cooking. It is also frequently used in place of lettuce on sandwiches. More recently chefs have begun offering watercress in salads and the red-pigmented watercress provides chefs and other food preparers with a striking color option when adding watercress to typically green salads. In addition, members of medical research communities in the U.S. and U.K. have begun detailing the varied anticancer properties of watercress.

The present invention provides a mutant allele designated “BWRW” which results in watercress plants having red-pigmented leaves and/or apical stems. The red pigmentation of the leaves can range in appearance from almost black to pink to purple depending on the environmental conditions whether one is looking at the upper or lower surface of the leaf. The range of colors include RHS N34A, RHS 45A, RHS 45B, RHS 45C, RHS 45D, RHS 46A, RHS 46B, RHS 46C, RHS 46D, RHS 47A, RHS 47B, RHS 47C, RHS 47D, RHS 53A, RHS 53B, RHS 53C, RHS 53D, RHS 58A, RHS 59A, RHS 59B, RHS 59C, RHS 60A, RHS 60B, RHS 60C, RHS 61A, RHS 63A, RHS 64A, RHS 64B, RHS 77A, RHS N77A, RHS N77B, RHS 79A, RHS 79B, RHS 79C, RHS 79D, RHS N79A, RHS N79B, RHS N79C, RHS N79D, RHS 83A, RHS N92A, RHS 181A, RHS 181B, RHS 181C, RHS 181D, RHS 182A, RHS 182B, RHS 182C, RHS 182D, RHS 183A, RHS 183B, RHS 183C, RHS 183D, RHS 184A, RHS 184B, RHS 184C, RHS 184D, RHS 185A, RHS 185B, RHS 185C, RHS 185D, RHS 186A, RHS 186B, RHS 186C, RHS 186D, RHS N186A, RHS N186B, RHS N186C, RHS N186D, RHS 187A, RHS 187B, RHS 187C, and RHS 187D.

In addition to conferring the red pigment to the leaves and/or apical stems, BWRW also affects the distribution of the pigment in the leaves depending on the environmental conditions. The red color is distributed mainly in the interveinal areas while the veins remain bright green under a short photoperiod (less than 10 h) or cold temperatures (under 44° F.). Under a long photoperiod (longer than 10 h) or warm temperatures (above 44° F.) the color is distributed largely at the leaf margins while the veins and the interveinal areas are bright green.

### Examples

The following examples are provided to further illustrate the present invention and are not intended to limit the invention beyond the limitations set forth in the appended claims.

### Example 1

### Development of BWRW, the Mutant Allele of the Present Invention

The mutant allele of the present invention, BWRW, unexpectedly arose as a spontaneous mutation in a population of triploid green watercress grown in New Market, Ala. Prior to the present invention, a red-pigmented leaf mutation of this type in watercress was unknown. The single, large, mutant plant was moved from Alabama to Fellsmere, Fla. and placed in a research bed. The line was expanded via stem cuttings to create an 8′×40′ bed. Additional expansion to commercial sized ponds of about 6 acres in size was accomplished via stem cuttings. In each 6-month period the line was uprooted and transferred between Alabama and Florida for further reproduction.

### Example 2

### Morphological Description

The red-pigmented watercress of the present invention has shown uniformity and stability for red-pigmented leaves and other traits, within the limits of environmental influence for the traits. The line has been increased with continued observation for uniformity. No variant traits have been observed or are expected in the present invention. The red-pigmented watercress of the present invention has the following morphologic and other characteristics.

TABLE 1
VARIETY DESCRIPTION

```
Plant: A triploid, perennial succulent temperate herb
Stems:
    Appearance: Floating, semi-erect, typically stays under water while 17.78 cm
    (7.0 in) turns upward and is harvested; roots freely at the axils
    Diameter: 0.635 cm (0.25 in) diameter at 17.145 cm (6.75 in) from apical
    meristem
    Internode length: 1.27 cm (0.5 in)
Leaves:
    Arrangement: A compound leaf with two opposite pair of oval leaflets and a
    pentagon shaped terminal leaflet
    Apex: Moderately apiculate
    Base: Inequilateral truncate to round
    Margin: Crenate to sinuate
    Color, under stress (44° F., or 10 hour photoperiod or less):
        Immature:
            Upper surface: RHS 186A (purple-grey) interveinal cells with
            RHS 142A (green) veins
            Lower surface: RHS 77A (purple) interveinal cells with RHS
            142A (green) veins
        Mature:
            Upper surface: RHS 186A (burgundy) throughout the interveinal
            area with RHS 142A (green) veins
            Lower surface: RHS 77A (purple) throughout the interveinal area
            with RHS 142A (green) veins
    Color, unstressed (above 44° F., or long day photoperiod): Leaf margins RHS
    186A, rest of tissue RHS 142A (green)
Inflorescence:
    Appearance: Raceme
    Floral cycle: One flower/seed production cycle per floral induction treatment
    Floral induction: At least 10 weeks of exposure to 36° F., followed by 30 days of
    growth at 72° F. and an 18 hour photoperiod
    Flower petal color: White
Fruit: Linear cylindrical silique
```

### Example 3

### Transfer of Mutant Allele to Other Genetic Backgrounds through Crossing

The present invention is transferred to other watercress genetic backgrounds through crossing with a diploid watercress line lacking the mutant allele of the present invention with the triploid red-pigmented watercress line carrying the mutant allele and screening for functional gametes. Plants subsequently produced are screened for those that are red-pigmented. The triploid red-pigmented watercress line carrying the mutant allele of the present invention is also crossed with an Xn watercress line lacking the mutant allele, where X=any number 1, 2, 3, 4, 5, or 6 to produce progeny plants carrying the mutant allele of the present invention and having red-pigmented leaves and/or apical stems. Marker-assisted breeding may be used at any point in the above breeding process.

### Example 4

### Transfer of Mutant Allele to Other Genetic Background through Doubling of Chromosomes

The present invention is transferred to other watercress genetic backgrounds through applying a chromosome doubling agent to a triploid red-pigmented watercress plant thus doubling the chromosome number from 3N to 6N. The hexaploid red-pigmented watercress plant is then crossed with any other hexaploid watercress plant lacking the mutant allele of the present invention. Progeny of the cross are screened for red-pigmented watercress plants. The hexaploid red-pigmented watercress plant is also crossed with any Xn watercress plant lacking the mutant allele of the present invention where X=any number 1, 2, 3, 4, 5, or 6 to produce progeny plants carrying the mutant allele of the present invention and having red-pigmented leaves and/or apical stems. Marker-assisted breeding may be used at any point in the above breeding process.

### Example 5

### Determination of Inheritance of the Mutant Allele of the Present Invention

The inheritance of the mutant allele of the present invention is determined by first determining the ploidy of the allele in red-pigmented watercress plants. Ploidy is determined by the use of flow cytometry. After ploidy is determined, crossing watercress plants of the present invention with watercress plants lacking the mutant allele allows for determination of segregation of the mutant allele and further defines inheritance of the mutant allele.

### Example 6

### Identification of Markers for the Mutant Allele of the Present Invention

At least one marker for the mutant allele of the present invention is identified using standard protocols. The identified markers are used to assess watercress plants for the allele of the present invention and to aid in breeding new watercress plants.

### Further Embodiments of the Invention

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes”. Over the last fifteen to twenty years several methods for producing transgenic plants have been developed and the present invention, in particular embodiments, also relates to transformed versions of the claimed variety or line.

Plant transformation involves the construction of an expression vector which will function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid and can be used alone or in combination with other plasmids to provide transformed watercress plants using transformation methods as described below to incorporate transgenes into the genetic material of the watercress plant(s).

### Expression Vectors for Watercress Transformation: Marker Genes

Expression vectors include at least one genetic marker operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. Fraley et al., Proc. Natl. Acad. Sci. USA, 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase and aminoglycoside-3′-adenyl transferase, the bleomycin resistance determinant (Hayford et al., Plant Physiol. 86:1216 (1988), Jones et al., Mol. Gen. Genet., 210:86 (1987), Svab et al., Plant Mol. Biol. 14:197 (1990), Hille et al., Plant Mol. Biol. 7:171 (1986)). Other selectable marker genes confer resistance to herbicides such as glyphosate, glufosinate or bromoxynil (Comai et al., Nature 317:741-744 (1985), Gordon-Kamm et al., Plant Cell 2:603-618 (1990) and Stalker et al., Science 242:419-423 (1988)).

Selectable marker genes for plant transformation not of bacterial origin include, for example, mouse dihydrofolate reductase, plant 5-enolpyruvylshikimate-3-phosphate synthase and plant acetolactate synthase (Eichholtz et al., Somatic Cell Mol. Genet. 13:67 (1987), Shah et al., Science 233:478 (1986), Charest et al., Plant Cell Rep. 8:643 (1990)).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include β-glucuronidase (GUS), β-galactosidase, luciferase and chloramphenicol acetyltransferase (Jefferson, R. A., Plant Mol. Biol. Rep. 5:387 (1987), Teeri et al., EMBO J. 8:343 (1989), Koncz et al., Proc. Natl. Acad. Sci. USA 84:131 (1987), DeBlock et al., EMBO J. 3:1681 (1984)).

In vivo methods for visualizing GUS activity that do not require destruction of plant tissue are available (Molecular Probes publication 2908, IMAGENE GREEN, pp. 1-4 (1993) and Naleway et al., J. Cell Biol. 115:151 a (1991)). However, these in vivo methods for visualizing GUS activity have not proven useful for recovery of transformed cells because of low sensitivity, high fluorescent backgrounds and limitations associated with the use of luciferase genes as selectable markers.

More recently, a gene encoding Green Fluorescent Protein (GFP) has been utilized as a marker for gene expression in prokaryotic and eukaryotic cells (Chalfie et al., Science 263:802 (1994)). GFP and mutants of GFP may be used as screenable markers.

### Expression Vectors for Watercress Transformation: Promoters

Genes included in expression vectors must be driven by a nucleotide sequence comprising a regulatory element, for example, a promoter. Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters that initiate transcription only in a certain tissue are referred to as “tissue-specific”. A “cell-type” specific promoter primarily drives expression in certain cell types in one or more organs, for example, vascular cells in roots or leaves. An “inducible” promoter is a promoter which is under environmental control. Examples of environmental conditions that may effect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions.

A. Inducible Promoters—An inducible promoter is operably linked to a gene for expression in watercress. Optionally, the inducible promoter is operably linked to a nucleotide sequence encoding a signal sequence which is operably linked to a gene for expression in watercress. With an inducible promoter the rate of transcription increases in response to an inducing agent.

Any inducible promoter can be used in the instant invention. See Ward et al., Plant Mol. Biol. 22:361-366 (1993). Exemplary inducible promoters include, but are not limited to, that from the ACEI system which responds to copper (Mett et al., Proc. Natl. Acad. Sci. USA 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone (Schena et al., Proc. Natl. Acad. Sci. USA 88:0421 (1991)).

B. Constitutive Promoters—A constitutive promoter is operably linked to a gene for expression in watercress or the constitutive promoter is operably linked to a nucleotide sequence encoding a signal sequence which is operably linked to a gene for expression in watercress.

Many different constitutive promoters can be utilized in the instant invention. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2: 163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)). The ALS promoter, Xba1/Nco1 fragment 5′ to the Brassica napus ALS3 structural gene (or a nucleotide sequence similarity to said Xba1/Nco1 fragment), represents a particularly useful constitutive promoter. See PCT application WO 96/30530.

C. Tissue-specific or Tissue-preferred Promoters—A tissue-specific promoter is operably linked to a gene for expression in watercress. Optionally, the tissue-specific promoter is operably linked to a nucleotide sequence encoding a signal sequence which is operably linked to a gene for expression in watercress. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter such as that from the phaseolin gene (Murai et al., Science 23:476-482 (1983) and Sengupta-Gopalan et al., Proc. Natl. Acad. Sci. USA 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Signal Sequences for Targeting Proteins to Subcellular Compartments

Transport of protein produced by transgenes to a subcellular compartment such as the chloroplast, vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine during protein synthesis and processing where the encoded protein is ultimately compartmentalized.

The presence of a signal sequence directs a polypeptide to either an intracellular organelle or subcellular compartment or for secretion to the apoplast. Many signal sequences are known in the art. See, for example, Becker et al., Plant Mol. Biol. 20:49 (1992); Close, P. S., Master's Thesis, Iowa State University (1993); Knox, C., et al., Plant Mol. Biol. 9:3-17 (1987); Lerner et al., Plant Physiol. 91:124-129 (1989); Frontes et al., Plant Cell 3:483-496 (1991); Matsuoka et al., Proc. Natl. Acad. Sci. 88:834 (1991); Gould et al., J. Cell. Biol. 108:1657 (1989); Creissen et al., Plant J. 2:129 (1991); Kalderon, et al., Cell 39:499-509 (1984); Steifel, et al., Plant Cell 2:785-793 (1990).

### Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to a preferred embodiment, the transgenic plant provided for commercial production of foreign protein is a watercress plant. In another preferred embodiment, the biomass of interest is seed. For the relatively small number of transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology, CRC Press, Boca Raton 269:284 (1993). Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and sequencing, all of which are conventional techniques.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

1. Genes that Confer Resistance to Pests or Disease and that Encode:

A. Plant disease resistance genes. Plant defenses are often activated by specific interaction between the product of a disease resistance gene (R) in the plant and the product of a corresponding avirulence (Avr) gene in the pathogen. A plant variety can be transformed with one or more cloned resistance genes to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al. Cell 78:1089 (1994) (Arabidopsis RSP2 gene for resistance to Pseudomonas syringae).

B. A gene conferring resistance to a pest, such as soybean cyst nematode. See e.g., PCT Application WO 96/30517; PCT Application WO 93/19181.

C. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995 and 31998.

D. A lectin. See, for example, Van Damme et al., Plant Molec. Biol. 24:25 (1994), who disclose the nucleotide sequences of several Clivia miniata mannose-binding lectin genes.

E. A vitamin-binding protein such as avidin. See PCT application US 93/06487 which teaches the use of avidin and avidin homologues as larvicides against insect pests.

F. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), Sumitani et al., Biosci. Biotech. Biochem. 57:1243 (1993) (nucleotide sequence of Streptomyces nitrosporeus α-amylase inhibitor) and U.S. Pat. No. 5,494,813 (Hepher and Atkinson, issued Feb. 27, 1996).

G. An insect-specific hormone or pheromone such as an ecdysteroid or juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

H. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata). See also U.S. Pat. No. 5,266,317 to Tomalski et al., which discloses genes encoding insect-specific, paralytic neurotoxins.

I. An insect-specific venom produced in nature by a snake, a wasp, etc. For example, see Pang et al., Gene 116:165 (1992), for disclosure of heterologous expression in plants of a gene coding for a scorpion insectotoxic peptide.

J. An enzyme responsible for a hyperaccumulation of a monoterpene, a sesquiterpene, a steroid, hydroxamic acid, a phenylpropanoid derivative or another non-protein molecule with insecticidal activity.

K. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 (Scott et al.), which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hornworm chitinase, and Kawalleck et al., Plant Molec. Biol. 21:673 (1993), who provide the nucleotide sequence of the parsley ubi4-2 polyubiquitin gene.

L. A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

M. A hydrophobic moment peptide. See PCT application WO 95/16776, which discloses peptide derivatives of tachyplesin which inhibit fungal plant pathogens, and PCT aapplication WO 95/18855 which teaches synthetic antimicrobial peptides that confer disease resistance.

N. A membrane permease, a channel former or a channel blocker. For example, see the disclosure of Jaynes et al., Plant Sci 89:43 (1993), of heterologous expression of a cecropin-β lytic peptide analog to render transgenic tobacco plants resistant to Pseudomonas solanacearum.

O. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-mediated resistance has been conferred upon transformed plants against alfalfa mosaic virus, cucumber mosaic virus and tobacco mosaic virus.

P. An insect-specific antibody or an immunotoxin derived therefrom. Thus, an antibody targeted to a critical metabolic function in the insect gut would inactivate an affected enzyme, killing the insect. See Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

Q. A virus-specific antibody. See, for example, Tavladoraki et al., Nature 366:469 (1993), who show that transgenic plants expressing recombinant antibody genes are protected from virus attack.

R. A developmental-arrestive protein produced in nature by a pathogen or a parasite. Thus, fungal endo-α-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

S. A developmental-arrestive protein produced in nature by a plant. For example, Logemann et al., Bio/Technology 10:305 (1992), have shown that transgenic plants expressing the barley ribosome-inactivating gene have an increased resistance to fungal disease.

T. Genes involved in the Systemic Acquired Resistance (SAR) Response and/or the pathogenesis-related genes. Briggs, S., Current Biology, 5(2) (1995).

U. Antifungal genes. See Cornelissen and Melchers, Plant Physiol., 101:709-712 (1993); Parijs et al., Planta 183:258-264 (1991) and Bushnell et al., Can. J. of Plant Path. 20(2):137-149 (1998).

V. Genes that confer resistance to Phytophthora root rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7 and other Rps genes. See, for example, Shoemaker et al., Phytophthora Root Rot Resistance Gene Mapping in Soybean, Plant Genome IV Conference, San Diego, Calif. (1995).

2. Genes that Confer Resistance to an Herbicide, for Example:

A. An herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively.

B. Glyphosate (resistance conferred by mutant 5-enolpyruvlshikimate-3-phosphate synthase (EPSPS) and aroA genes, respectively) and other phosphono compounds such as glufosinate (phosphinothricin acetyl transferase (PAT) and Streptomyces hygroscopicus PAT bar genes), and pyridinoxy or phenoxy proprionic acids and cyclohexones (ACCase inhibitor-encoding genes). See, for example, U.S. Pat. No. 4,940,835 to Shah, et al., which discloses the nucleotide sequence of a form of EPSPS which can confer glyphosate resistance. A DNA molecule encoding a mutant aroA gene can be obtained under ATCC accession number 39256, and the nucleotide sequence of the mutant gene is disclosed in U.S. Pat. No. 4,769,061 to Comai. European patent application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European application No. 0 242 246 to Leemans et al. DeGreef et al., Bio/Technology 7:61 (1989) describe the production of transgenic plants that express chimeric bar genes coding for phosphinothricin acetyl transferase activity. Exemplary of genes conferring resistance to phenoxy proprionic acids and cyclohexones, such as sethoxydim and haloxyfop are the Acc1-S1, Acc1-S2, and Acc2-S3 genes described by Marshall et al., Theor. Appl. Genet. 83:435 (1992).

C. An herbicide that inhibits photosynthesis, such as a triazine (psbA and gs+ genes) and a benzonitrile (nitrilase gene). Przibila et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Nos. 53435, 67441 and 67442. Cloning and expression of DNA coding for a glutathione S-transferase is described by Hayes et al., Biochem. J. 285:173 (1992).

D. Acetohydroxy acid synthase, which has been found to make plants that express this enzyme resistant to multiple types of herbicides, has been introduced into a variety of plants. See Hattori et al., Mol. Gen. Genet. 246:419, 1995. Other genes that confer tolerance to herbicides include a gene encoding a chimeric protein of rat cytochrome P4507A1 and yeast NADPH-cytochrome P450 oxidoreductase (Shiota et al., Plant Physiol., 106:17, 1994), genes for glutathione reductase and superoxide dismutase (Aono et al., Plant Cell Physiol. 36:1687, 1995), and genes for various phosphotransferases (Datta et al., Plant Mol. Biol. 20:619, 1992).

E. Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288, 306; 6,282,837; 5,767,373; and international publication WO 01/12825.

3. Genes that Confer or Contribute to a Value-Added Trait, such as:

A. Modified fatty acid metabolism, for example, by transforming a plant with an antisense gene of stearyl-ACP desaturase to increase stearic acid content of the plant. See Knultzon et al., Proc. Natl. Acad. Sci. USA 89:2625 (1992).

B. Decreased phytate content—1) Introduction of a phytase-encoding gene enhances breakdown of phytate, adding more free phosphate to the transformed plant. For example, see Van Hartingsveldt et al., Gene 127:87 (1993), for a disclosure of the nucleotide sequence of an Aspergillus niger phytase gene. 2) A gene could be introduced that reduced phytate content. This could be accomplished by cloning and then reintroducing DNA associated with the single allele which is responsible for maize mutants characterized by low levels of phytic acid. See Raboy et al., Maydica 35:383 (1990).

C. Modified carbohydrate composition effected, for example, by transforming plants with a gene coding for an enzyme that alters the branching pattern of starch. See Shiroza et al., J. Bacteriol. 170:810 (1988) (nucleotide sequence of Streptococcus mutants fructosyltransferase gene), Steinmetz et al., Mol. Gen. Genet. 20:220 (1985) (nucleotide sequence of Bacillus subtilis levansucrase gene), Pen et al., Bio/Technology 10:292 (1992) (production of transgenic plants that express Bacillus lichenifonnis α-amylase), Elliot et al., Plant Molec. Biol. 21:515 (1993) (nucleotide sequences of tomato invertase genes), Søgaard et al., J. Biol. Chem. 268:22480 (1993) (site-directed mutagenesis of barley a-amylase gene), and Fisher et al., Plant Physiol. 102:1045 (1993) (maize endosperm starch branching enzyme II).

D. Elevated oleic acid via FAD-2 gene modification and/or decreased linolenic acid via FAD-3 gene modification. See U.S. Pat. Nos. 6,063,947; 6,323,392; and international publication WO 93/11245.

4. Genes that Control Male Sterility:

A. Introduction of a deacetylase gene under the control of a tapetum-specific promoter and with the application of the chemical N-Ac-PPT. See international publication WO 01/29237.

B. Introduction of various stamen-specific promoters. See international publications WO 92/13956 and WO 92/13957.

C. Introduction of the barnase and the barstar genes. See Paul et al., Plant Mol. Biol. 19:611-622, 1992).

### Methods for Watercress Transformation

Numerous methods for plant transformation have been developed including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc. Boca Raton, 1993) pp. 67-88. In addition, expression vectors and in-vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

### A. Agrobacterium-Mediated Transformation

One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra and Moloney et al., Plant Cell Reports 8:238 (1989). See also, U.S. Pat. No. 5,563,055 (Townsend and Thomas), issued Oct. 8, 1996.

### B. Direct Gene Transfer

Several methods of plant transformation, collectively referred to as direct gene transfer, have been developed as an alternative to Agrobacterium-mediated transformation. A generally applicable method of plant transformation is microprojectile-mediated transformation where DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5:27 (1987); Sanford, J. C., Trends Biotech. 6:299 (1988); Klein et al., Bio/Tech. 6:559-563 (1988); Sanford, J. C. Physiol Plant 7:206 (1990); Klein et al., Biotechnology 10:268 (1992). See also U.S. Pat. No. 5,015,580 (Christou, et al.), issued May 14, 1991 and U.S. Pat. No. 5,322,783 (Tomes, et al.), issued Jun. 21, 1994.

Another method for physical delivery of DNA to plants is sonication of target cells. Zhang et al., Bio/Technology 9:996 (1991). Alternatively, liposome and spheroplast fusion have been used to introduce expression vectors into plants. Deshayes et al., EMBO J., 4:2731 (1985); Christou et al., Proc Natl. Acad. Sci. USA 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described (Donn et al., In Abstracts of VIIth International Congress on Plant Cell and Tissue Culture IAPTC, A2-38, p 53 (1990); D'Halluin et al., Plant Cell 4:1495-1505 (1992) and Spencer et al., Plant Mol. Biol. 24:51-61 (1994)).

Following transformation of watercress target tissues, expression of the above-described selectable marker genes allows for preferential selection of transformed cells, tissues and/or plants, using regeneration and selection methods well known in the art.

The foregoing methods for transformation would typically be used for producing a transgenic variety. The transgenic variety could then be crossed with another (non-transformed or transformed) variety in order to produce a new transgenic variety. Alternatively, a genetic trait that has been engineered into a particular watercress line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simple X by Y cross or the process of backcrossing depending on the context.

### Gene Conversions

When the term “watercress plant” is used in the context of the present invention, this also includes any single gene conversions of that variety. The term gene converted plant as used herein refers to those watercress plants which are developed by a plant breeding technique called backcrossing wherein essentially all of the desired morphological and physiological characteristics of a variety are recovered in addition to the one or more genes transferred into the variety via the backcrossing technique. Backcrossing methods can be used with the present invention to improve or introduce at least one characteristic into the variety. The term “backcrossing” as used herein refers to the repeated crossing of a hybrid progeny back to the recurrent parent, i.e., backcrossing 1, 2, 3, 4, 5, 6, 7, 8 or more times to the recurrent parent. The parental watercress plant that contributes the one or more genes for the desired characteristics is termed the nonrecurrent or donor parent. This terminology refers to the fact that the nonrecurrent parent is used one time in the backcross protocol and therefore does not recur. The parental watercress plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original variety of interest (recurrent parent) is crossed to a second variety (nonrecurrent parent) that carries the gene(s) of interest to be transferred. The resulting progeny from this cross are then crossed again to the recurrent parent and the process is repeated until a watercress plant is obtained wherein essentially all of the desired morphological and physiological characteristics of the recurrent parent are recovered in the converted plant, in addition to the transferred gene(s) from the nonrecurrent parent.

The selection of a suitable recurrent parent is an important step for a successful backcrossing procedure. The goal of a backcross protocol is to alter or substitute one or more traits or characteristics in the original variety. To accomplish this, at least one gene of the recurrent variety is modified or substituted with the desired gene(s) from the nonrecurrent parent, while retaining essentially all of the rest of the desired genetic, and therefore the desired physiological and morphological, constitution of the original variety. The choice of the particular nonrecurrent parent will depend on the purpose of the backcross; one of the major purposes is to add some agronomically important trait to the plant. The exact backcrossing protocol will depend on the characteristic(s) or trait(s) being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic(s) has/have been successfully transferred.

Many traits have been identified that are not regularly selected for in the development of a new variety but that can be improved by backcrossing techniques. These traits may or may not be transgenic; examples of these traits include but are not limited to, male sterility, herbicide resistance, resistance for bacterial, fungal, or viral disease, insect resistance, male fertility, enhanced nutritional quality, industrial usage, yield stability and yield enhancement. These genes are generally inherited through the nucleus. Several of these traits are described in U.S. Pat. Nos. 5,959,185; 5,973,234 and 5,977,445; the disclosures of which are specifically hereby incorporated by reference for this purpose.

### Tissue Culture

Further reproduction of a variety can occur by tissue culture and regeneration. Tissue culture of various tissues of watercress and regeneration of plants therefrom is well known and widely published. For example, reference may be had to Jelaska, S. et al., Physiol. Plant. 64(2):237-242 (1985) and Krsnik-Rasol, M., Int. J. Dev. Biol. 35(3):259-263 (1991). Thus, another aspect of this invention is to provide cells which upon growth and differentiation produce watercress plants having the mutant allele BWRW.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, pods, leaves, stems, roots, root tips, anthers, pistils and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

### Additional Breeding Methods

This invention also is directed to methods for producing a watercress plant by crossing a first parent watercress plant with a second parent watercress plant wherein the first or second parent watercress plant is a watercress plant comprising the mutant allele BWRW. Further, both first and second parent watercress plants can comprise the mutant allele BWRW. Thus, any such methods using a watercress plant comprising the mutant allele BWRW are part of this invention: selfing, backcrosses, hybrid production, crosses to populations, and the like.

### Tables

Watercress plants grow as long indeterminate stems which may elongate in a prostrate or upright orientation. Prostrate forms usually root into support media at the axils while simultaneously producing an axil stem which orients upright for harvest. Upright forms bend upright forming an L shape without long prostrate runs. Yields of the prostrate form are usually greater than upright forms assuming an equal density of original “transplanted stems”. Unexpectedly, the red watercress plants containing the BWRW mutant allele of the present invention present an intermediate form, grow more slowly than standard green watercress, and produce 36% fewer marketable stem bunches than standard 3n or 2n green watercress lines. Table 2 shows the yield of red watercress plants containing the BWRW mutant allele of the present invention as compared to the yield of standard green watercress plants. In Table 2, column 1 shows the type of watercress, column 2 shows the ploidy, column 3 shows the year, columns 4 through 12 show the yield in bunches per acre and column 13 shows the mean yield for each year.

TABLE 2
Comparison of Red Watercress Containing the BWRW Mutant Allele with
Standard Green Watercress Lacking the BWRW Mutant Allele for Yield over Two Years

| | | | Four Week Periods Beginning January 1 | | | | | | | | | |
|---|---|---|---|---|---|---|---|---|---|---|---|---|
| Watercress Line | Ploidy | Year | 1 | 2 | 3 | 4 | 5 | 6 | 11 | 12 | 13 | Mean |
| Red Watercress | 3N | 2008 | 18,800 | 12,770 | 16,529 | 7,336 | 16,592 | 15,771 | | 15,520 | 11,919 | 14,405 |
| | | 2009 | 15,750 | 13,730 | 19,226 | 15,270 | 8,996 | | | | | 14,594 |
| Green | 3N | 2008 | 21,732 | 23,823 | 25,354 | 22,850 | 27,303 | 22,990 | 21,495 | 21,223 | 18,795 | 22,841 |
| Watercress USA | | | | | | | | | | | | |
| Standard | | | | | | | | | | | | |
| | | 2009 | 16,151 | 25,353 | 22,588 | 23,979 | 25,228 | 23,196 | | | | 22,749 |
| Green | 2N | 2008 | 18,257 | 18,782 | 20,862 | 21,674 | 26,828 | 21,234 | | 15,808 | 17,800 | 20,156 |
| Watercress UK | | | | | | | | | | | | |
| Standard | | | | | | | | | | | | |
| | | 2009 | 27,433 | 26,269 | 18,401 | 20,982 | | | | | | 23,271 |

As shown in Table 2, the red watercress containing the mutant allele BWRW of the present invention unexpectedly yielded significantly fewer bunches per acre than either the standard U.S. or U.K. green watercresses which lack the BWRW mutant allele. Importantly, while slower growth reduces the total number of bunches and crops per season, slower growth allows the crop to hold longer in the field before going out of market specifications. An additional benefit is that individual stems do not develop as large an undesirable central hole as faster growing lines do.

Watercress stem diameters affect how watercress is used by consumers. Watercress can be consumed fresh as a lettuce substitute or in a mix of fresh greens; it can be boiled, sautéed or stir-fried. Watercress lines marketed for fresh consumption typically possess a smaller stem diameter, for example in the U.K. where watercress is most often consumed fresh, the standard stem diameter is 2.33 mm. Watercress lines marketed for stir fry, boiling, or sautéing possess larger stem diameters of 3 to 6 mm. Unexpectedly, the red watercress containing the BWRW mutant allele of the present invention had a mean stem diameter that was intermediate between the green standard U.S. watercress line and the green standard U.K. watercress line.

Table 3 shows the mean stem number per bunch, the mean weight per stem, and the mean stem diameter for red watercress which contains the BWRW mutant allele of the present invention as compared to standard U.S. and U.K. green watercresses which lack the BWRW mutant allele. Also included are two experimental green watercresses, 47-8 and 96-1, which lack the BWRW mutant allele of the present invention. In Table 3 column 1 shows the watercress line, column 2 shows the ploidy of each line, column 3 shows the growing location, column 4 shows the date the watercress was cut and the measurements taken, column 5 shows the mean stem number per bunch, column 6 shows the mean weight in grams per stem, column 7 shows the mean stem diameter in millimeters, and column 8 shows the standard deviation of the stem diameter in millimeters.

TABLE 3
Comparison of Various Stem Characteristics between Red Watercress Containing the BWRW Mutant Allele and Standard
Green Watercress and Two Experimental Watercress Lines

| | | | | Mean Stem # | Mean Wt per | Mean Stem | Stem Diameter, |
|---|---|---|---|---|---|---|---|
| Watercress Line | Ploidy | Location | Cut Date | per Bunch | Stem (g) | Diameter (mm) | Std Dev (mm) |
| Red Watercress | 3N | Florida | Jan. 12, 2009 | 34 | 4.68 | 4.39 | 1.11 |
| Red Watercress | 3N | Tennessee | Jun. 5, 2009 | 49 | 4.14 | 3.58 | 1.26 |
| Green Watercress US | 3N | Florida | Jan. 12, 2009 | 42 | 3.59 | 3.60 | 1.09 |
| Standard | | | | | | | |
| Green Watercress US | 3N | Tennessee | Jun. 5, 2009 | 45 | 4.63 | 4.06 | 1.22 |
| Standard | | | | | | | |
| Green Watercress | 2N | Florida | Jan. 14, 2009 | 91 | 1.57 | 2.33 | 0.76 |
| UK Standard | | | | | | | |
| 47-8 | 6N | Florida | Jan. 12, 2009 | 44 | 3.48 | 3.39 | 1.02 |
| 47-8 | 6N | Tennessee | Jun. 5, 2009 | 32 | 6.64 | 5.37 | 1.27 |
| 96-1 | 6N | Florida | Jan. 12, 2009 | 28 | 5.99 | 4.46 | 1.11 |
| 96-1 | 6N | Tennessee | Jun. 5, 2009 | 48 | 4.86 | 3.86 | 1.10 |

As shown in Table 3, the red watercress stem diameter, 3.58 to 4.39 mm, compares favorably with the U.S. standard 3n line, 3.6 to 4.06 mm, and with two new experimental hexaploid lines. However, the red watercress containing the BWRW mutant allele of the present invention unexpectedly has significantly fewer stems per bunch than the standard U.K. green watercress which lacks the mutant allele.

While significantly more watercress is sold during the winter holiday period in the U.S. when cooler temperatures favor high yields and larger stem growth, watercress lines that do not produce reasonably large diameter stems in the higher temperatures of summer will not be kept in a commercial rotation for long. For example, the U.K. standard is not grown in the U.S. during the summer because its stem diameter is too small. In contrast, the red watercress containing the BWRW mutant allele of the present invention unexpectedly can be grown in the U.S. in summer because its stem diameter of about 3.58 mm is commercially acceptable for a variety of uses by consumers. The stem diameter of the red watercress containing the BWRW allele of the present invention is such that it is not too small to be used for stir-fry, boiling and sautéing nor is it too big to be used for fresh consumption. As shown in Table 3, the BWRW allele allows watercress to be grown year-round unlike the standard green U.K. watercress.

The assay Ferric Reducing Ability of Plasma (FRAP) is considered an assay of antioxidant power. In tests at the University of South Hampton, U.K., watercress FRAP values of a number of watercress lines were tested and ranged from 1.61 to 5.65 nmol Fe2+ equivalents per gram fresh weight. In these tests red watercress containing the BWRW mutant allele of the present invention had an unexpected FRAP value of 5.65 nmol which is at least 2.27 times greater than the mean of 2.27 nmol Fe2+ equivalents per gram fresh weight generated for green watercress lines which lack the BWRW mutant allele. This means the red watercress has greater antioxidant power than standard green watercress lines.

Selfed seed has been produced from 3N red watercress containing the BWRW mutant allele of the present invention. Watercress seed germination generally varies with ploidy, with 2N, 4N, and 6N green lines producing germination rates of ˜87% or better. However, the germination rate of selfed red watercress seed containing the BWRW mutant allele of the present invention is typically 47%.

Red watercress containing the BWRW mutant allele of the present invention has been successfully crossed to diploid green leaf watercress forms which lack the BWRW mutant allele and the red leaf trait has been observed in various F₁ and F₂ progeny. Because the trait is not observed in all F₁ plants the data suggest that the mutation occurred in only one of the two genomes that make up 3n red watercress. Red watercress containing the BWRW mutant allele of the present invention was crossed to green watercress 3-0291, a proprietary 2N watercress and in another trial was crossed to green watercress 3-0420, also known as Improved Large Leaf, which is a 2N commercial watercress line from England. The F₁ population segregated for ploidy, red leaf color, and leaf size/shape. The 10 best F₂ seedlings having the red leaf color and containing the BWRW mutant allele of the present invention have been given the numbers 3-0654 to 3-0663 and are continuing to be tested in a watercress breeding program.

### Deposit Information

A deposit of the watercress seed and tissue of this invention is maintained by B&W Quality Growers, Inc., 17825 79th Street, Fellsmere, Fla. 32948-7822. Access to this deposit will be available during the pendency of this application to persons determined by the Commissioner of Patents and Trademarks to be entitled thereto under 37 CFR §1.14 and 35 USC §122. Upon allowance of any claims in this application, all restrictions on the availability to the public of the variety will be irrevocably removed by affording access to a deposit of at least 2,500 seeds of the same variety with the American Type Culture Collection, Manassas, Va. or National Collections of Industrial, Food and Marine Bacteria (NCIMB), 23 St Machar Drive, Aberdeen, Scotland, AB24 3RY, United Kingdom or to a deposit of at least 25 vials of tissue culture of the same variety with International Patent Organism Depositary, AIST Tsukuba Central 6, 1-1-1 Higashi, Tsukuba, Ibaraki, Japan or with DSMZ-Deutsche Sanunlung von Mikro-organismen and Zellkulturen GmbH, Inhoffenstralβe 7 B, 38124 Braunschweig, Germany.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A watercress seed containing an allele designated BWRW.

2. A watercress plant, or a part thereof, produced by growing said watercress seed of claim 1.

3. A watercress seed containing an allele designated BWRW, wherein a representative sample of seed containing said allele BWRW was deposited under ATCC Accession No. ______.

4. A tissue culture of cells containing an allele designated BWRW, wherein a representative sample of cells containing said allele BWRW was deposited under ATCC Accession No. ______.

5. A tissue culture of cells produced from the plant of claim 2, wherein said cells of the tissue culture are produced from a plant part selected from the group consisting of leaf, pollen, embryo, cotyledon, hypocotyl, meristematic cell, root, root tip, pistil, anther, flower, stem, and petiole.

6. A protoplast produced from the plant of claim 2.

7. A protoplast produced from the tissue culture of claim 5.

8. A watercress plant regenerated from the tissue culture of claim 5.

9. A watercress plant regenerated from the tissue culture of claim 4.

10. A method for producing a watercress seed, wherein the method comprises crossing the plant of claim 2 with a different watercress plant and harvesting the resultant watercress seed.

11. A watercress seed produced by the method of claim 10.

12. A watercress plant, or a part thereof, produced by growing said seed of claim 11.

13. A method of producing an herbicide resistant watercress plant, wherein the method comprises transforming the watercress plant of claim 2 with a transgene wherein the transgene confers resistance to an herbicide selected from the group consisting of imidazolinone, sulfonylurea, glyphosate, glufosinate, L-phosphinothricin, triazine and benzonitrile.

14. An herbicide resistant watercress plant produced by the method of claim 13.

15. A method of producing an insect resistant watercress plant, wherein the method comprises transforming the watercress plant of claim 2 with a transgene that confers insect resistance.

16. An insect resistant watercress plant produced by the method of claim 15.

17. The watercress plant of claim 16, wherein the transgene encodes a Bacillus thuringiensis endotoxin.

18. A method of producing a disease resistant watercress plant, wherein the method comprises transforming the watercress plant of claim 2 with a transgene that confers disease resistance.

19. A disease resistant watercress plant produced by the method of claim 18.

20. A method of producing a watercress plant with modified fatty acid metabolism or modified carbohydrate metabolism, wherein the method comprises transforming the watercress plant of claim 2 with a transgene encoding a protein selected from the group consisting of fructosyltransferase, levansucrase, α-amylase, invertase and starch branching enzyme or encoding an antisense of stearyl-ACP desaturase.

21. A watercress plant having modified fatty acid metabolism or modified carbohydrate metabolism produced by the method of claim 20.
